(19) United States
(12) Patent Application Publication
(10) Pub. No.: US 2011/0287492 A1
(43) Pub. Date: Nov. 24, 2011

(54) METHOD FOR THE REDUCTION OF REPETITIVE SEQUENCES IN ADAPTER-LIGATED RESTRICTION FRAGMENTS

(75) Inventors: Daniele Trebbi, Wageningen (IT); Michael Josephus Theresia van Eijk, Wageningen (NL)

(73) Assignee: Keygene N.V.

(21) Appl. No.: 13/132,698

(22) PCT Filed: Nov. 25, 2009
(86) PCT No.: PCT/NL2009/000232

§ 371 Date: Aug. 4, 2011

Related U.S. Application Data
(60) Provisional application No. 61/119,864, filed on Dec. 4, 2008.

(51) Int. Cl. C12P 19/34

(52) U.S. Cl. 435/91.52

(57) ABSTRACT

The present invention relates to a method for the reduction of repetitive sequences (or the improvement in low-copy sequences) in DNA samples by a combination of restriction endonuclease treatment, followed by adapter ligation, renaturation kinetics-based fractionation, optionally coupled with duplex sequence nucleases and further restriction endonuclease treatment, followed by adapter ligation. The low-copy enriched fractions can be used in further DNA analysis.

The present invention relates to methods for reducing the amount repetitive sequences in genomes and to methods for enriching samples for low-copy sequences. The invention further relates to the use of renaturation kinetics based fractionation for the enrichment of adapter-ligated restriction fragments for low-copy sequences. The invention further relates to the use of double strand-specific nuclease (DSN) for the enrichment of adapter-ligated restriction fragments for low-copy sequences. The invention further relates to the use of samples enriched for low-copy fragments in fingerprint analysis, SNP mining and marker development.

### BACKGROUND OF THE INVENTION

Many genomes, in particular plants, contain repetitive sequences, i.e. parts, whether large or smaller, of the DNA that are repeated at several positions in a genome. In particular when genome sizes increase and in particular in plants, where genomes can have large sizes (e.g. Arabidopsis thaliana 130 Mb, Oryza sativa 430 Mb, Zea Mays 2400Mb, Barley 5300 Mb and Onion 15000 Mb per haploid genome). Some genomes such as pepper and maize are said to contain up to 80% of repetitive sequences.

AFLP (EP534858, Vos et al 1995) is a successful technology for the development of markers and the identification of SNPs that are linked to a particular trait or QTL. To subsequently develop such AFLP markers or SNPs to PCR markers, i.e. markers that can be used with a normal PCR assay, the markers or SNPs have to be converted. The conversion is a laborious process that in many occasions is not successful (Brugmans et al. Nucleic Acids Research 2003 31(10):e55) in particular due to the presence of repetitive sequences in the genome. Repetitive sequences do not yield useful markers or SNPs that can be linked to a trait or a quantitative trait locus. That in itself does not necessarily pose problem, but the presence of repetitive sequences hampers the subsequent conversion of AFLP markers to PCR markers. Therefore, there is a need to enrich restriction fragment mixtures obtained from DNA samples for low-copy sequences, because this will increase the probability for successful conversion to PCR markers and thereby improve the effectiveness of genetic marker development for traits

### SUMMARY OF THE INVENTION

The present inventors have now found that a combination of elements of the AFLP technology, DNA reassociation kinetics and DSN normalisation provides an efficient and reliable way of enriching a DNA sample for low copy sequences. The enrichment for low copy sequences is achieved, according to the invention by providing adapter-ligated restriction fragments from a DNA sample using a first restriction endonuclease, subjecting the adapter-ligated restriction fragments to renaturation kinetics based fractionation and subjecting the fractions to a DSN, restricting the remaining fragments with a second restriction endonuclease and ligating a second adapter to the restriction fragment to thereby obtain a low-copy enriched fraction of the starting DNA.

### Definitions

In the following description and examples, a number of terms are used. In order to provide a clear and consistent understanding of the specification and claims, including the scope to be given such terms, the following definitions are provided. Unless otherwise defined herein, all technical and scientific terms used have the same meaning as commonly understood by one of ordinary skill in the art to which this invention belongs. The disclosures of all publications, patent applications, patents and other references are incorporated herein in their entirety by reference.

AFLP: AFLP refers to a method for selective amplification of nucleic acids based on digesting a nucleic acid with one or more restriction endonucleases to yield restriction fragments, ligating adaptors to the restriction fragments and amplifying the adaptor-ligated restriction fragments with at least one primer that is (part) complementary to the adaptor, (part) complementary to the remains of the restriction endonuclease, and that further contains at least one randomly selected nucleotide from amongst A, C, T, or G (or U as the case may be) at the 3′end of the primer. AFLP does not require any prior sequence information and can be performed on any starting DNA. In general, AFLP comprises the steps of:

(a) digesting a nucleic acid, in particular a DNA or cDNA, with one or more specific restriction endonucleases, to fragment the DNA into a corresponding series of restriction fragments;

(b) ligating the restriction fragments thus obtained with a double-stranded synthetic oligonucleotide adaptor, one end of which is compatible with one or both of the ends of the restriction fragments, to thereby produce adaptor-ligated, restriction fragments of the starting DNA;

(c) contacting the adaptor-ligated, restriction fragments under hybridizing conditions with one or more oligonucleotide primers that contain selective nucleotides at their 3′-end;

(d) amplifying the adaptor-ligated, restriction fragment hybridised with the primers by PCR or a similar technique so as to cause further elongation of the hybridised primers along the restriction fragments of the starting DNA to which the primers hybridised; and

(e) detecting, identifying or recovering the amplified or elongated DNA fragment thus obtained.

AFLP thus provides a reproducible subset of adaptor-ligated fragments. AFLP is described in EP 534858, U.S. Pat. No. 6,045,994 and in Vos et al 1995. AFLP: a new technique for DNA fingerprinting. Nucleic Acids Research 23(21):4407-4414. Reference is made to these publications for further details regarding AFLP. The AFLP is commonly used as a complexity reduction technique and a DNA fingerprinting technology. Within the context of the use of AFLP as a fingerprinting technology, the concept of an AFLP marker has been developed.

AFLP marker: An AFLP marker is an amplified adaptor-ligated restriction fragment that is different between two samples that have been amplified using AFLP (fingerprinted), using the same set of primers. As such, the presence or absence of this amplified adaptor-ligated restriction fragment can be used as a marker that is linked to a trait or phenotype. With conventional slab gel electrophoresis , an AFLP marker shows up as a band in the gel with a certain mobility. Other electrophoretic techniques such as capillary electrophoresis may not refer to this as a band, but the concept remains the same, i.e. a nucleic acid with a certain length and mobility. Absence or presence of the band may be indicative of (or associated with) the presence or absence of the phenotype. AFLP markers typically involve polymorphisms in the restriction site of the endonuclease (type 1) or the in selective nucleotides (type 2). Occasionally, AFLP markers may involve indels (insertion-deletions in the restriction fragment (type 3).

Selective base or selective nucleotide: Located at the 3′ end of the primer that contains a part that is complementary to the adaptor and a part that is complementary to the remains of the restriction site, the selective base is randomly selected from amongst A, C, T or G (or U as the case may be). By extending a primer with a selective base, the subsequent amplification will yield only a reproducible subset of the adaptor-ligated restriction fragments, i.e. only the fragments that can be amplified using the primer carrying the selective base. Selective nucleotides can be added to the 3′end of the primer in a number varying between 1 and 10. Typically, 1-4 suffice. Both primers may contain a varying number of selective bases. With each added selective base, the subset reduces the amount of amplified adaptor-ligated restriction fragments in the subset by a factor of about 4. Typically, the number of selective bases used in AFLP is indicated by +N/+M, wherein one primer carries N selective nucleotides and the other primers carries M selective nucleotides. Thus, an EcoRI/Msel +1/+2 AFLP is shorthand for the digestion of the starting DNA with EcoRI and MseI, ligation of appropriate adaptors and amplification with one primer directed to the EcoRI restricted position carrying one selective base and the other primer directed to the MseI restricted site carrying 2 selective nucleotides. A primer used in AFLP that carries at least one selective nucleotide at its 3′ end is also depicted as an AFLP-primer. Primers that do not carry a selective nucleotide at their 3′ end and which in fact are complementary to the adaptor and the remains of the restriction site are sometimes indicated as AFLP+0 primers. The term selective nucleotide is also used for nucleotides of the target sequence that are located adjacent to the adaptor section and that have been identified by the use of selective primer as a consequence of which, the nucleotide has become known.

Sequencing: The term sequencing refers to determining the order of nucleotides (base sequences) in a nucleic acid sample, e.g. DNA or RNA. Many techniques are available such as Sanger sequencing and high-throughput sequencing technologies such as offered by 454 technologies (Roche Applied Science), IIlumina Inc. and Applied BioSystems, Helicos and others.

Restriction endonuclease: a restriction endonuclease or restriction enzyme is an enzyme that recognizes a specific nucleotide sequence (target site) in a double-stranded DNA molecule, and will cleave both strands of the DNA molecule at or near every target site, leaving a blunt or a staggered end.

Frequent cutters and rare cutters: Restriction enzymes typically have recognition sequences that vary in number of nucleotides from 3, 4 (such as MseI) to 6 (EcoRI) and even 8 (NotI). The restriction enzymes used can be frequent and rare cutters. The term ‘frequent’ in this respect is typically used in relation to the term ‘rare’. Frequent cutting endonucleases (aka frequent cutters) are restriction endonucleases that have a relatively short recognition sequence. Frequent cutters typically have 3-5 nucleotides that they recognise and subsequently cut. Thus, a frequent cutter on average cuts a DNA sequence every 64-512 nucleotides. Rare cutters are restriction endonucleases that have a relatively long recognition sequence. Rare cutters typically have 6 or more nucleotides that they recognise and subsequently cut. Thus, a rare 6-cutter on average cuts a DNA sequence every 1024 nucleotides, leading to longer fragments. It is observed again that the definition of frequent and rare is relative to each other, meaning that when a 4 bp restriction enzyme, such as MseI, is used in combination with a 5-cutter such as AvaII, AvaII is seen as the rare cutter and MseI as the frequent cutter.

Restriction fragments: the DNA molecules produced by digestion with a restriction endonuclease are referred to as restriction fragments. Any given genome (or nucleic acid, regardless of its origin) will be digested by a particular restriction endonuclease into a discrete set of restriction fragments. The DNA fragments that result from restriction endonuclease cleavage can be further used in a variety of techniques and can for instance be detected by gel electrophoresis.

Ligation: the enzymatic reaction catalyzed by a ligase enzyme in which two double-stranded DNA molecules are covalently joined together is referred to as ligation. In general, both DNA strands are covalently joined together, but it is also possible to prevent the ligation of one of the two strands through chemical or enzymatic modification of one of the ends of the strands. In that case the covalent joining will occur in only one of the two DNA strands.

Synthetic oligonucleotide: single-stranded DNA molecules having preferably from about 10 to about 50 bases, which can be synthesized chemically are referred to as synthetic oligonucleotides. In general, these synthetic DNA molecules are designed to have a unique or desired nucleotide sequence, although it is possible to synthesize families of molecules having related sequences and which have different nucleotide compositions at specific positions within the nucleotide sequence. The term synthetic oligonucleotide will be used to refer to DNA molecules having a designed or desired nucleotide sequence.

Adaptors: short double-stranded DNA molecules with a limited number of base pairs, e.g. about 10 to about 30 base pairs in length, which are designed such that they can be ligated to the ends of restriction fragments. Adaptors are generally composed of two synthetic oligonucleotides which have nucleotide sequences which are partially complementary to each other. When mixing the two synthetic oligonucleotides in solution under appropriate conditions, they will anneal to each other forming a double-stranded structure. After annealing, one end of the adaptor molecule is designed such that it is compatible with the end of a restriction fragment and can be ligated thereto; the other end of the adaptor can be designed so that it cannot be ligated, but this need not be the case (double ligated adaptors).

Adaptor-ligated restriction fragments: restriction fragments that have been capped by adaptors.

Primers: in general, the term primers refer to synthetic DNA molecules which can prime the synthesis of DNA. DNA polymerase cannot synthesize DNA de novo without a primer: it can only extend an existing DNA strand in a reaction in which the complementary strand is used as a template to direct the order of nucleotides to be assembled. We will refer to the synthetic oligonucleotide molecules which are used in a polymerase chain reaction (PCR) as primers.

DNA amplification: the term DNA amplification will be typically used to denote the in vitro multiplication of DNA molecules, using PCR yielding double stranded DNA or other other amplification methods such as GenomiPhi/RepliG yielding single stranded DNA. It is noted that yet other amplification methods exist and they may be used in the present invention without departing from the gist.

Repetitive sequence: a repetitive sequence is a sequence that is repeated many times, but at least two or more times in a DNA or a genome. Highly-repetitive sequence: a highly-repetitive sequence is a sequence that is repeated typically hundreds to thousands of times in a DNA or a genome. Examples are Tandem repeats such as satellite DNA, minisatellites and microsatellite and Interspersed repeats such as SINEs (Short INterspersed Elements) and LINEs (Long INterspersed Elements), (retro)transposons.

Low-copy sequence, low copy-number sequence, unique sequence: sequence that occurs only in a limited number of copies (or only in one copy) in a genome and can be used as a marker linked to a phenotype. Low-copy fraction: fraction of a DNA sample that is reduced in repetitive sequences and hence is enriched in low-copy sequences, i.e. a fraction from which repetitive sequences have been removed.

DSN (double strand-specific nuclease): a nuclease that specifically cuts double stranded DNA sequences (Shagin et al 2002 Genome Research 12 1935-1945, Zhulidov et al 2004 NAR 32 3 e37).

Renaturation kinetics based fractionation: a technique wherein low-copy sequences are separated from high-copy or repetitive sequences in a DNA sample . The procedure involves heating a sample of genomic DNA until it denatures into the single stranded-form, and then slowly cooling it, so the strands can pair back together. While the sample is cooling, measurements can be taken of how much of the DNA is base paired at each temperature.

The amount of single and double-stranded DNA can be measured by rapidly diluting the sample, which slows reassociation, and then binding the DNA to a hydroxylapatite column. The column can be eluted to elute the single-stranded DNA first and followed by elution of the double stranded DNA. The amount of DNA in these two solutions can be measured using a spectrophotometer. Since a sequence of single-stranded DNA needs to find its complementary strand to reform a double helix, common sequences renature more rapidly than rare sequences. Indeed, the rate at which a sequence will reassociate is proportional to the number of copies of that sequence in the DNA sample. A sample with a highly-repetitive sequence will renature rapidly, while complex sequences will renature slowly.

However, instead of simply measuring the percentage of double-stranded DNA versus time, the amount of renaturation is usually measured relative to a C₀t value. The C₀t value is the product of C₀ (the initial concentration of DNA), t (time in seconds), and a constant that depends on the concentration of cations in the buffer. Repetitive DNA will renature at low C₀t values, while complex and unique DNA sequences will renature at high C₀t values.

Cₒt filtration: Cₒt filtration is a technique that uses the principles of DNA renaturation kinetics to separate the repetitive DNA sequences that dominate many eukaryotic genomes from “gene-rich” single/low-copy sequences. This allows DNA sequencing to concentrate on the parts of the genome that are most informative and interesting, which will speed up the discovery of new genes and make the process more efficient (Peterson D G, Wessler S R, Paterson A H (2002). “Efficient capture of unique sequences from eukaryotic genomes”. Trends Genet. 18 (11): 547-50, LLamoureux D, Peterson D G, Li W, Fellers J P, Gill B S (2005). “The efficacy of Cₒt-based gene enrichment in wheat (Triticum aestivum L.)”. Genome 48 (6): 1120-6. , Yuan Y, SanMiguel P J, Bennetzen J L (2003). “High-Cₒt sequence analysis of the maize genome”. Plant J. 34 (2): 249-55.

### DETAILED DESCRIPTION OF THE INVENTION

Thus, in a first embodiment the invention relates to a method for the reduction of repetitive sequences (enhancing the (relative) amount of low-copy sequences) in adapter-ligated restriction fragments comprising the steps of:
a. restricting a starting DNA in a sample with a first restriction endonuclease and ligating a first adapter to the restriction fragments to obtain adapter-ligated restriction fragments;
b. performing a renaturation kinetics based fractionation on the adapter-ligated restriction fragments obtained in step (a);
c. subjecting the renatured fractions of step (b) to a duplex specific nuclease (DSN);
d. restricting the restriction fragments obtained from step (c) with a second restriction endonuclease and ligating a second adapter to the restriction fragments;
e. obtaining a low-copy enriched fraction of the starting DNA.

The method according to the invention combines certain elements of AFLP (creation of adapter-ligated restriction fragments using a restriction enzyme and ligation of adapters) with certain elements of renaturation kinetics based fracturation (allowing high copy sequences to renature to become double stranded but low copy sequences to remain single-stranded), removing the double stranded sequences by DSN and, again AFLP (further creation of adapter-ligated restriction fragments using a second restriction enzyme. The results is that a fraction of the starting DNA is obtained that is enriched for the presence of low-copy sequences and reduced in the number of highly repetitive sequences.

In step (a) of the method according to the invention, a starting DNA is treated with a restriction endonuclease. The starting DNA can be any DNA such as genomic DNA, cDNA, BAC DNA, mitochondrial DNA, chloroplast DNA or mixtures of plant genomic DNA containing mitochondrial and chloroplast DNA. The first restriction endonuclease digests the starting DNA to yield restriction fragments. The first restriction endonuclease is preferably a rare cutter, such as a hexacutter, such as EcoRI, EcoRII, BamHI, HindIII, PstI leading to restriction fragments which typically range in size between 1-4 kb in case an AT-rich endonuclease such as EcoRI (recognition sequence GAATTC) is applied in and AT-rich organism such as most plant species. To these restriction fragments, first adapters are ligated to yield adapter-ligated restriction fragments.

In step (b) of the method according to the invention, the adapter-ligated restriction fragments, are subjected to a renaturation kinetics-based fractionation. Thus, the adapter-ligated restriction fragments are heated until denaturation into the single stranded-form, and then slowly cooled down, so the strands can pair back together. As the adapter-ligated restriction fragments are typically within the same length range due to the use of the resection endonuclease, the repetitive sequences will re-anneal within a determinable amount of time while the low-copy sequences will remain single stranded for a longer period of time. After cooling has proceeded long enough, the re-annealing is stopped, for instance by forced cooling. In renaturation kinetics-based fractionation disclosed in the art, the amount of single and double-stranded DNA is usually measured by rapidly diluting the sample, which slows reassociation, and then binding the DNA to a hydroxylapatite column after which the single stranded and double stranded fractions can be eluted.

In a preferred embodiment of the invention, the use of the column and the intermediate separation into fractions that essentially contain single stranded DNA or double stranded DNA, respectively, can be avoided by (directly) treating the sample with a double stranded nuclease in step (c) of the method. The double stranded nuclease specifically cuts double stranded DNA sequences (Shagin et al 2002 Genome Research 12 1935-1945, Zhulidov et al 2004 NAR 32 3 e37). As most of the repetitive sequences are now in double stranded form, the sample is reduced for adapter-ligated restriction fragments that are derived from repetitive sequences (or enriched for adapter-ligated restriction fragments that are derived from low-copy or unique sequences).

The renatured fractions of step (b), whether or not DSN treated in optional step (c), are subjected in step (d) of the method of the invention to a second digestion with a restriction endonuclease, followed by ligation of the adapter corresponding to the endonuclease. The resulting adapter-ligated restriction fragments, that, in a preferred embodiment, on each side of the restriction fragment carry a different adapter. Thus the result of the method of the current invention is that a set of adapter-ligated restriction fragments is obtained that is enriched for low-copy DNA (or reduced significantly in relative occurrence of repetitive DNA).

The obtained adapter-ligated restriction fragments can be used in subsequent fingerprinting technologies such as AFLP, for SNP mining etc.. Thus in one embodiment, the obtained low-copy DNA sample can be selectively amplified and fingerprinted to obtain higher quality fingerprints.

The Cₒt methodology is in itself well known for the normalisation of cDNA libraries, also in combination with DSN. Typically, Cₒt-fragments are obtained by shearing or otherwise by fragmenting DNA in a more or less random manner. Prior to Cₒt analysis, fragments are typically size selected to reduce the effect that larger fragments renature quicker than shorter fragments, which could be detrimental to the desired result. By now performing a first endonuclease restriction and adapter ligation before Cₒt treatment and further fragmentation, the size of the fragments are more manageable and generally lie within a more limited size range. On the other hand, performing endonuclease restriction using two enzymes (and hence two adapters), would lead to a too large spread of lengths (in the area of 900-50 bp). By executing the method with a first restriction step, then a Cₒt treatment and a subsequent restriction step, the Cₒt treatment is performed on a set of adapter-ligated restriction fragments that have renaturation kinetics that are more homogeneous than those of fragment mixtures containing a wide range of large and small adapter-ligated fragments.

The method of the present invention is particularly useful to increase the success rate of conversion of AFLP markers (or other markers) to, for instance PCR markers and in the discovery of useful SNPs which can be turned into assays applicable on whole genomic DNA.

### DESCRIPTION OF THE FIGURES

FIG. 1 Pepper results for Pepper E33/M49 and Pepper P14/M60 are

FIG. 2 Maize results for Maize E35/M48 and Maize P12/M50

FIG. 3 Cₒt-DSN Protocol

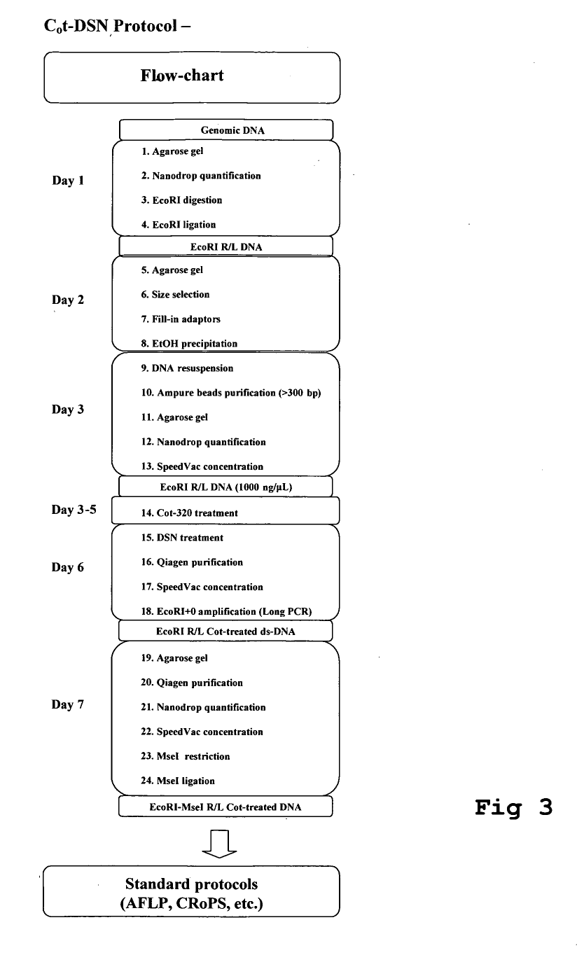

### EXAMPLES

The invention will now be explained by the following examples. It will be clear to the skilled man that variations on this protocol are possible without departing from the gist of the invention,
1. Agarose gel
   For DNA quality/integrity check, load 5 μl DNA (RNAse treated!) on a 1% agarose gel.
2. Nanodrop quantification
   For accurate quantification of DNA, dry and resuspend/Speedvacuum concentrate the DNA to 1000 ng/μL if necessary (minimum concentration of 114 ng/μL)
3. EcoRI DNA digestion (60 μg DNA)

| Reagent | Conc. | Sample | |
|---|---|---|---|
| 60 μg DNA | 1000 ng/μL | 60 | μL |
| EcoRI | 50 U/μL | 6 | μL |
| One-Phor-All | 10x | 60 | μL |
| BSA | 10 mg/μL | 3 | μL |
| DTT | 1M | 3 | μL |
| Milli Q water | | 468 | μL |
| Total volume | | 600 | μL |

Treat samples at 37° C. for 6 hours.

Use 5 U of EcoRI per μg DNA [standard AFLP protocol requires 25 U/μg DNA]

Amount of DNA can vary from a minimum of 20 μg to more than 100 μg (scalable)

Size-selection step between restriction and ligation is not performed
4. EcoRI adapter ligation

| Reagent | Conc. | Sample |
|---|---|---|
| EcoRI Adaptor | 5 μM | 300 |
| One-Phor-All | 10x | 100 |
| BSA | 10 mg/μL | 2 |
| DTT | 1M | 2 |
| ATP | 10 mM | 300 |
| T4 ligase | 1 U/μL | 300 |
| Total volume | | 1004 |

The 1004 μL are added to the restricted 600 μL and treated at 37° C. overnight.

Use 40 ng DNA/pmole adaptor [like in the standard AFLP protocol]
5. Agarose gel
   Check quality of the EcoRI digestion. Expect a smear between 1-10 kb with highest intensity at 2-3 kb.
6. Size-selection
   Treat samples at 80° C. for 20 min. in a water-bath.
7. Fill-in adaptors (ds adaptors synthesis)
   In order to make ds-adaptor the following 300 μL solution is added to each tube containing 1.604 mL R/L

| Reagent | Conc. | Sample |
|---|---|---|
| 10xPCR buffer | ROCHE | 190 |
| dNTPs | 5 mM | 70 |
| Taq Polymerase | ROCHE 5 U/μL | 7 |
| Milli Q water | | 33 |
| Total volume (1x PCR) | | 300 |

Treat samples (1.904 mL) at 72° C. for 30 min. in a water-bath, then chill tubes on ice and EtOH precipitate.
8. DNA EtOH precipitation of ds-RL samples
   Transfer the 1.904 mL R/L in a 50 mL tube (blue cap, V bottom).
   Add the same volume (1.9 mL) of 7.5 M Ammonium Acetate and mix.
   Add three volumes (11.4 mL) of 100% ethanol and let DNA precipitate at −20° C. overnight.
   Centrifuge samples at 4800 rpm (large rotor) for 20 min. at 4° C.

Wash pellet with 1.0 mL cold 70% ethanol and carefully (!) transfer the pellet into a 1.5 mL eppendorf tube.

Centrifuge tubes at 14000 rpm (micro centrifuge) for 20 minutes at 4° C.
Carefully discard supernatant and let EtOH evaporate from pellets at 37° C. for 15-20 minutes.
9. DNA resuspension
   Re-suspend the pellet in 100 μL Milli Q water or EB buffer (QIAGEN) leaving tubes at RT for 3 hours or at 60° C. for 20 min. and mixing slowly by pipetting.
10. Ampure Bead Purification of fragments>300 bp (See Ampure Protocol)
   At the 100 μL of the resuspended samples add 400 μL EB buffer (QIAGEN) and mix.
   Add 350 μL of Ampure beads and vortex (Ratio Beads/Sample=0.7 to have cutoff˜300 bp).
   Incubate samples for 5 min at RT.
   Insert tubes in the (Magnetic Particle Concentrator) MPC for 5 min. to precipitate the beads.
   Remove supernatant by pipetting (leave tubes in the MPC).
   Keep tubes in the MPC and wash beads with 1 mL 70% EtOH and discard supernatant (twice).
   Dry pellets at 37° C. for 10-15 minutes.
   Remove tubes from the MPC and elute DNA with 200 μL Milli Q water (twice) by adding Milli Q water, vortexing/pipetting to resuspend all beads and leaving tubes in MPC for 5 min for beads precipitation.
   Keep the supernatant (with DNA!) and place it in a new 1.5 mL tube (400 μL/sample).
11. Agarose gel
   Check quality of purification (load 2μL on a 1% agarose gel).
12. Nanodrop quantification
   For accurate DNA quantification.
13. SpeedVac concentration to 1000 ng/μL
   Samples are concentrated to obtain ds-R/L purified DNA at a concentration of 1000 ng/μL.
   (Alternatively dry the sample and re-suspend it in the correct volume of Milli Q water).
14. Cₒt treatment
   Cₒt treatment is performed at the following conditions:
   1. 500 ng/μL DNA

### 2. 0.3 M NaCl

3. renaturation at 63□C
At these conditions Cₒt-320 takes 60 h 16 m
Hybridization is performed as follow (in 200μL tubes) :

Sample

DNA (at 1000 ng/μL) 2 μL

### 2× Cₒt Hybridization Buffer (0.6 M NaCl)

Note: composition −30% of 4× trimmer hybridization buffer at 2 M NaCl −70% MQW 2μL

Total volume 4 μL
Cover the 4 μL sample with 20 μL mineral oil and centrifuge (make sure the 4 μL are covered by oil).
Treat samples at 95° C. for 10 min (PCR thermocycler) and let hybridize at 63° C. for the time required for the Cₒt treatment (Cₒt-320=60 h 16 m).
15. DSN treatment
   Preheat the DSN Master Buffer at 63° C. for>5 min.
   Cₒt treatment is stopped (after 60 h 16 m 00 s) placing the sample on ice for 30 seconds.
   Place samples at 63° C. for 1 min.
   Add 5 μL of preheated DSN Master Buffer to the 4 μL of renaturating solutions, mix quickly by pipetting and incubate at 63° C. for additional 10 min.
   Add 1 μL DSN Enzyme, mix quickly by pipetting and centrifuge (make sure all the 10 μL are on the bottom of the tube) and treat at 68° C. for 25 min.
   Add 10μL of Stop Solution, mix and centrifuge and keep at 68° C. for additional 5 min before placing tubes on ice.
   Add 20 μL sterile water [total volume after DSN treatment=40 μL] and purify the sample.
16. Qiagen purification
   Follow Qiagen purification instruction and elute in 150 μL Milli Q water (three 50 μL elutions).
17. SpeedVac concentration
   Concentrate the 150μL to 31 μL (˜40 min. at 35° C.). Adjust volume to 31 μL if necessary.
18. EcoRI+0 ds-synthesis of the DSN-treated ss-DNA samples using Advantage 2 PCR kit
   30 μL (of the 31 μL eluted from Qiagen purification) is +0 amplified to make ds-DNA as follow.
   The remaining 1 μL (Not +0 amplified) will be used for agarose analysis

Sample

| | |
|---|---|
| Purified ss DNA from DSN treatment | 30 |
| EcoRI + 0 EOOL (50 ng/μL) | 1.5 |
| 10x Ad2 buffer | 5 |
| 50x dNTP | 1 |
| 50x Ad2 Polymerase mix | 1 |
| MQW | 11.5 |
| total | 50 |

The samples are treated at 94° C. for 30 sec. then 24 cycles at 94° C. for 7 sec., 56° C. for 30 sec. and 72° C. for 10 min., with last step at 72° C. for 10 min and stored at 4° C.
19. Agarose gel
   Load 1 μL of “Not +0 amplified” and 1.6 μL “+0 amplified” for each sample.
20. Qiagen purification
   Follow Qiagen purification instruction and elute in 150 μL Milli Q water (three 50 μL elutions).
21. Speedvacuum concentration
   Concentrate the 150μL to 40-50 μL (minimum DNA concentration of 14 ng/μL).
22. Nanodrop quantification
   For accurate DNA quantification.
23. Msel Restriction (Standard AFLP protocol)

| Reagent | Conc. | Sample |
|---|---|---|
| Cₒt treated DNA | (500 ng) + Milli Q water | 35.6 |
| Msel | 40 U/μL | 0.03 |
| One-Phor-All | 10x | 4 |
| BSA (μL) | 10 mg/μL | 0.2 |
| DTT (μL) | 1M | 0.2 |
| Total volume | | 40 |

Samples are treated at 37° C. for 1.0 h

Size-selection step between restriction and ligation is not performed
24. Msel adapter Ligation (Standard AFLP protocol)

| Reagent | Conc. | Sample |
|---|---|---|
| Msel Adapter | 50 μM | 1 |
| One-Phor-All | 10x | 1 |
| BSA | 10 mg/μL | 0.05 |
| DTT | 1M | 0.05 |
| ATP | 10 mM | 1 |
| T4 ligase | 1 U/μL | 1 |
| Milli Q Water | | 5.9 |
| Total volume | | 10 |

The 10 μL are added to the restricted 40 μL and treated at 37° C. overnight.
Starting from the Cₒt-treated R/L samples, the standard protocols (i.e. for AFLP, CRoPS, etc. can be followed)
The reduction of repetitive sequences was performed by selecting samples from pepper and maize (both highly repeated genomes). The composition of convention R/L mixes (restriction-ligation mixtures) prior to any Cₒt treatment was

| | |
|---|---|
| EcoRI/Msel | 100-1000 bp |
| Pstl/Msel | 100-1000 bp |
| Msel-only with partial digestion | 1000 bp average |
| EcoRI-only | 2000-3000 bp average |
| Pstl-only | 4000-5000 bp average |

Cₒt parameter ranging from 0-320 (0, 1, 2, 5, 10, 20, 40, 80, 160, 320)

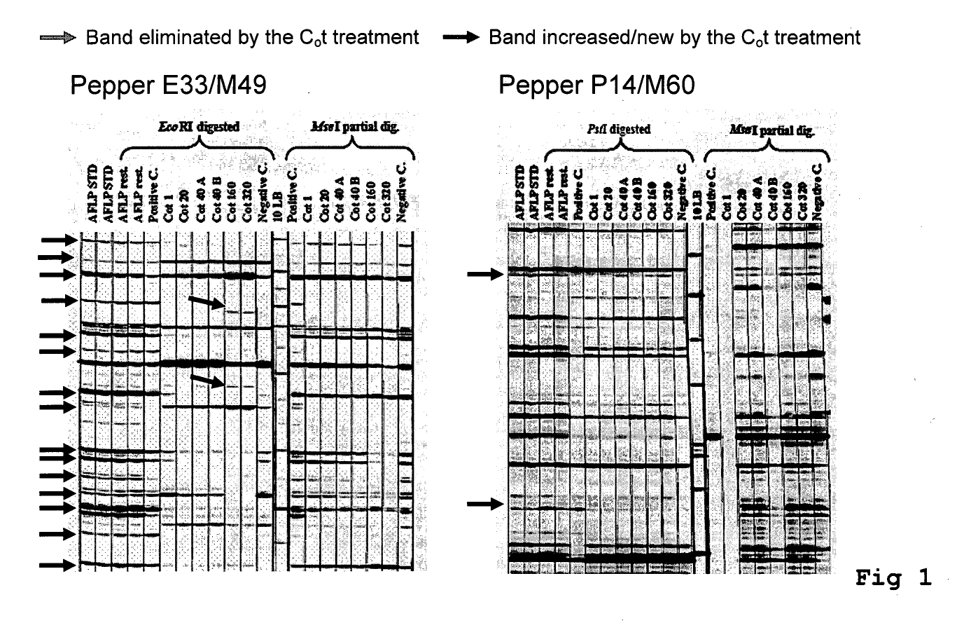

Pepper results for Pepper E33/M49 and Pepper P14/M60 are displayed in FIG. 1.

The various restriction enzyme digests and Cₒt denaturation times were tested on two pepper samples that were restricted with EcoRI (pepper E33/M49) and amplified using primer E33 (E-AAG) and M49 (M-CAG) and with PstI (pepper P14/M60) and amplified using primer P14 (P-AT) and M60 (M-CTC). From FIG. 1 it is clear that repetitive forms are reduced and some hitherto undiscovered low-copy bands appear

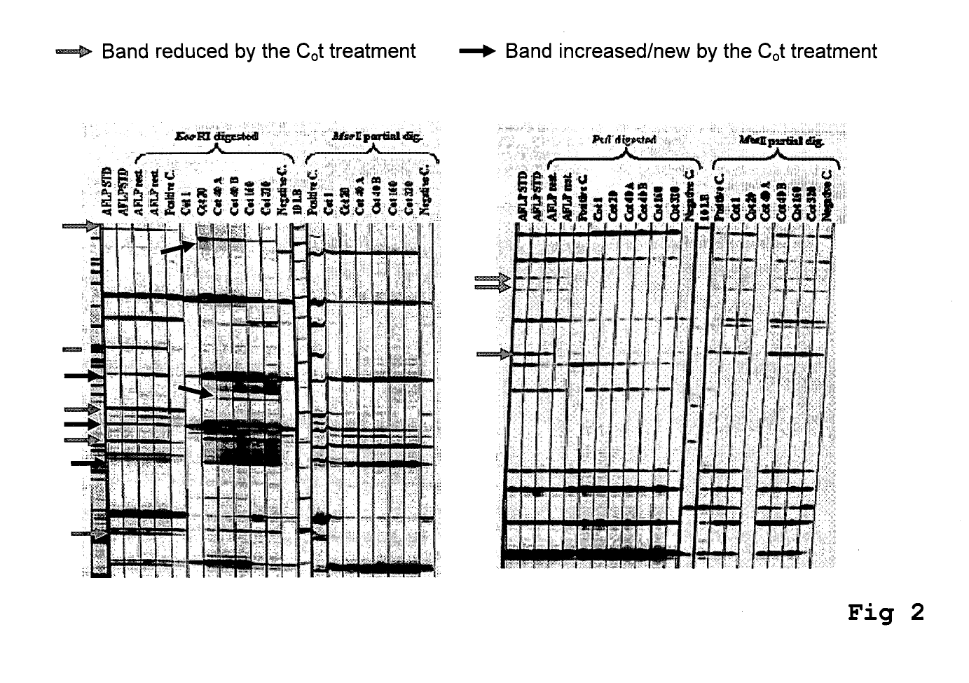

Maize results for Maize E35/M48 and Maize P12/M50 are displayed in FIG. 2.

The various restriction enzyme digests and Cₒt denaturation times were tested on two Maize samples that were restricted with EcoRI (Maize E35/M48) and amplified using primer E35 (E-ACA) and M48 (M-CAC) and with PstI (Maize P12/M50) and amplified using primer P12 (P-AC) and M50 (M-CAT). From FIG. 2 it is clear that repetitive forms are reduced and some hitherto undiscovered low-copy bands appear

From these results it was concluded that

| Pre-treatment | Cₒt effect |
|---|---|
| EcoRI/Msel digestion | No |
| Pstl/Msel digestion | No |
| Msel-partial | No |
| EcoRI only | Yes |
| Pst-only | Yes |
| Cₒt time | 320 |

| | | | | | No. variable bands |
|---|---|---|---|---|---|
| | | | | % Reduction | by Cₒt |
| | No. Bands | No. bandsat | New bandsat | of bands by | treatment(variable |
| | AFLP std. | Cₒt 320 | Cₒt 320 | Cₒt treatment | intensity) |
| Pepper E35/M48 | 82 | 34 | 0 (0%) | 58% | 9 (27%) |
| Pepper E33/M49 | 81 | 49 | 3 (4%) | 43% | 16 (33%) |
| Maize E35/M48 | 39 | 30 | 3 (8%) | 31% | 9 (30%) |
| Maize E33/M49 | 30 | 18 | 2 (7%) | 47% | 3 (17%) |
| Cₒt effect | | | ~5% | 40 to 50% | 20 to 30% |

These results indicate that the Cₒt treatment determined the disappearance of 40-50% of the standard AFLP bands and the appearance of 5% on new bands. A total of 20-30% of the bands that remained after the Cₒt treatment presented a variation (increased or decreased) of band intensity.

Validation on qRT-PCR Pepper

| | STS single | Ribosomal | Chloroplast |
|---|---|---|---|
| | copy gene | repetitive | repetitive |
| gDNA | 1 | 47.8 | 45.1 |
| R/L before treatment (EcoRI- | 1 | 49.8 | 43.8 |
| only) | | | |
| Normalized gDNA-No Cₒt | 1 | 60.5 | 67.4 |
| Normalized gDNA-Cₒt 320 | 1 | 13.6 | 23.7 |
| Cot effect | | −4.5 fold | −2.9 fold |
| R/L post treatment-No Cₒt | 1 | 13.5 | 34.1 |
| R/L post treatment-Cₒt 320 | 1 | 5.4 | 5 |
| Cot effect | | −2.5 fold | −6.8 fold |
| +1/+2-No Cₒt | 1 | 67.0 | 115.9 |
| +1/+2-Cₒt 320 | 1 | 16.3 | 41.8 |
| Cₒt effect | | −4.1 fold | −2.8 fold |

From these results is concluded that sequences that are ˜40 fold more abundant than single copy genes are reduced ˜4 fold by the Cₒt treatment (40:1 to 10:1)

Further validation is achieved by Sanger sequencing.

1. A Method for the reduction of repetitive sequences (enhancing the (relative) amount of low-copy sequences) in adapter-ligated restriction fragments comprising the steps of:
a. restricting a starting DNA in a sample with a first restriction endonuclease and ligating a first adapter to the restriction fragments to obtain adapter-ligated restriction fragments;
b. performing a renaturation kinetics based fractionation on the adapter-ligated restriction fragments obtained in step (a);
c. optionally, subjecting the renatured fractions of step (b) to a duplex specific nuclease (DSN);
d. restricting the restriction fragments obtained from step (b) or (c) with a second restriction endonuclease and ligating a second adapter to the restriction fragments;
e. obtaining a low-copy enriched fraction of adapter-ligated restriction fragments from the starting DNA.

2. The method according to claim 1, wherein the first restriction endonuclease is a rare cutter.

3. The method according to claim 2, wherein the second restriction endonuclease is a frequent cutter.

4. The method according to claim 3, wherein the renaturation kinetics based fractionation is Cₒt

5. The method according to claim 4, wherein the Cₒt value is 320

6. The method according to claim 5, wherein the low-copy fraction of the adaptor-ligated restriction fragments obtained from starting DNA is amplified using primers directed to the adapters having optionally 1-10 selective nucleotides at the 3′end

7. The method according to claim 6, wherein the starting DNA is genomic DNA, cDNA, BAC DNA, mitochondrial DNA, chloroplast DNA or mixtures of plant genomic DNA with mitochondrial DNA and chloroplast DNA

8. The method according to claim 6 wherein DSN normalisation is performed using a treatment comprising a duplex specific nuclease.

9. The production of AFLP fingerprints enriched for low-copy sequences, according to the method of claim 1.

10. A method of treating a DNA sample prior to marker conversion or SNP mining, comprising the method for the reduction of repetitive sequences of claim 1.
